(19) United States
(12) Patent Application Publication
(10) Pub. No.: US 2023/0029032 A1
(43) Pub. Date: Jan. 26, 2023

(54) IN-ACTIVITY VISUALIZATIONS IN A DISTRIBUTED ENVIRONMENT

(71) Applicant: ECHELON FITNESS MULTIMEDIA LLC, Chattanooga, TN (US)

(72) Inventors: Lou LENTINE, Stuart, FL (US); John SANTO, Orlando, FL (US)

(21) Appl. No.: 17/377,552

(22) Filed: Jul. 16, 2021

(51) Int. Cl. A63B 24/00; H04N 5/76; A63B 71/06; A63B 22/00; A63B 22/06; G16H 20/30

(52) U.S. Cl. CPC A63B 24/0084; H04N 5/76; A63B 24/0062; A63B 71/0622; A63B 22/0076; A63B 22/06; G16H 20/30; A63B 2071/0675; A63B 2071/0625; A63B 2220/806; A63B 2225/20

(57) ABSTRACT

The disclosed embodiments are directed toward improvements in user interfaces of fitness devices. In one embodiment, an improved user interface (UI) is disclosed whereby controls for adjusting the physical metrics of a fitness device are conveniently located. In another embodiment, a multimedia content functionality is described wherein users of fitness devices can record multimedia content and associate that multimedia content with metrics associated with exercise activity. In another embodiment, a fitness device is capable of streamlining multimedia content during the performance of an activity and can adjust multimedia streams during such an activity.

### BACKGROUND

The disclosed embodiments are directed toward fitness equipment and devices and, specifically, toward the generation of leaderboard visualizations and other user interfaces in a geographically dispersed activity environment.

Traditionally, many fitness activities were undertaken as in-person endeavors. Spin, rowing, cycling, and other classes required the physical presence of participants to coordinate an activity under the guidance of a single teacher. As home exercise equipment became widely available, some users replaced or augmented fitness routines with personal (e.g., in-home, in-office, etc.) fitness equipment such as exercise bikes and rowing machines. However, this early equipment frequently provided rudimentary interfaces that only allowed users to see their current activity (e.g., speed, power, etc.).

More recently, network-enabled fitness equipment has allowed personal fitness equipment to “connect” to other network-enabled equipment. Such systems could allow users to virtually race one other while being physically dispersed. However, such systems still rely on rudimentary visualizations such as static leaderboards that fail to leverage the wealth of data generated in a distributed fitness activity.

### BRIEF SUMMARY

The disclosed embodiments solve these and other problems in the current state of the art. The disclosed embodiments describe a distributed fitness environment whereby activity metrics recorded locally by individual fitness equipment or devices are synchronized with a central cache or database. The fitness device generates these activity metrics during live classes or during archived classes. In a live class, a user of the fitness device competes with other users of other fitness equipment. In an archived class, the user of the fitness device competes with past users of a class or activity. In some embodiments, the past users may include or consist of the user themselves; that is, the past users may include a user's previous engagement with the class or activity.

A publish-subscribe architecture is provided whereby activity metrics are published and consumed by the fitness device. Thus, fitness equipment receives raw data representing the current state of the users participating in an activity. In turn, the fitness device can then generate visualizations of the raw data for display on a screen of the fitness device.

In one embodiment, an improved user interface (UI) is disclosed whereby controls for adjusting the physical metrics of a fitness device are conveniently located. In another embodiment, a multimedia content functionality is described wherein users of fitness devices can record multimedia content and associate that multimedia content with metrics associated with exercise activity. In another embodiment, a fitness device is capable of streamlining multimedia content during the performance of an activity and can adjust multimedia streams during such an activity.

Further detail on various embodiments including additional embodiments are described in more detail below.

### BRIEF DESCRIPTION OF THE DRAWINGS

The preceding and other objects, features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure.

FIG. 1A is a user interface diagram of a user interface (UI) displayed on a fitness device according to some embodiments of the disclosure.

FIGS. 1B, 1C, and 1D are user interface diagrams of Uls displayed on a fitness device according to some embodiments of the disclosure.

FIG. 2 is a user interface diagram of a leaderboard displayed on a fitness device according to some embodiments of the disclosure.

FIG. 3 is a user interface diagram of the main window displayed on a fitness device according to some embodiments of the disclosure.

FIG. 4 is a flow diagram illustrating a method for generating a visualization on a fitness device according to some embodiments of the disclosure.

FIG. 5 is a flow diagram illustrating a method for propagating activity data according to some embodiments of the disclosure.

FIG. 6 is a block diagram of a system 600 for distributing and visualizing leaderboard data according to some embodiments of the disclosure.

FIG. 7 is a block diagram illustrating a computing device showing an example of a client or server device used in the various embodiments of the disclosure.

FIG. 8A is a flow diagram illustrating a method for capturing and recording a user image during an activity according to some embodiments of the disclosure.

FIG. 8B is a flow diagram illustrating a method for generating a year in review multimedia object according to some embodiments of the disclosure.

FIG. 9A is a flow diagram illustrating a method for adjusting a metric level according to some embodiments of the disclosure.

FIG. 9B is a flow diagram illustrating a method for adjusting a metric level according to some embodiments of the disclosure.

FIG. 10 is a flow diagram illustrating a method for displaying streaming media content during an activity according to some embodiments of the disclosure.

### DETAILED DESCRIPTION

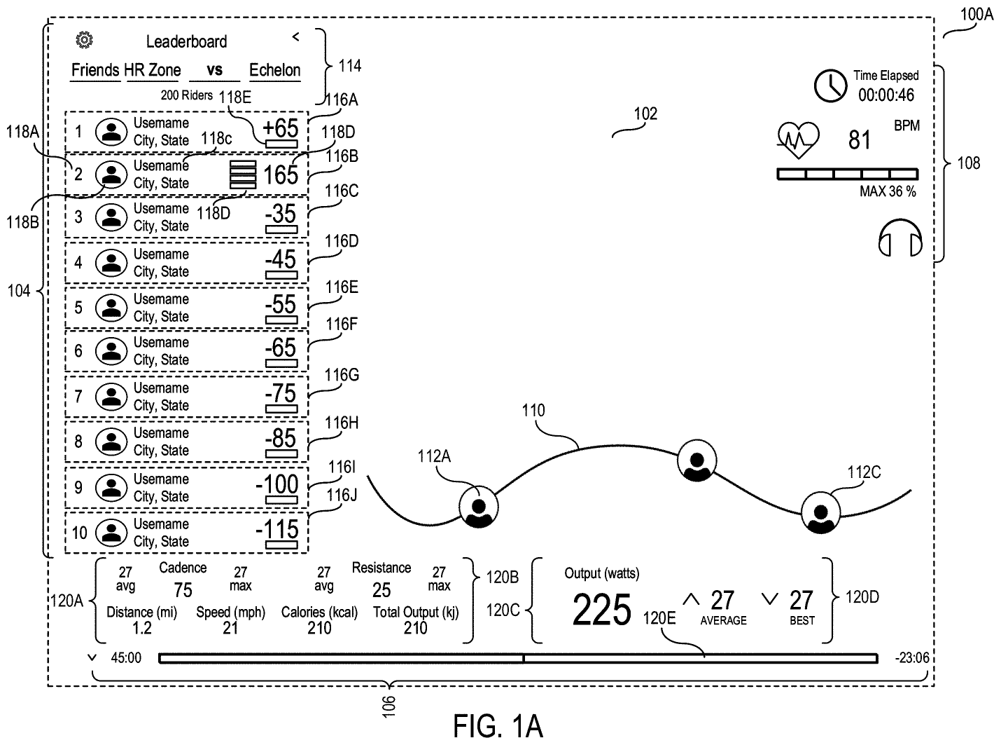

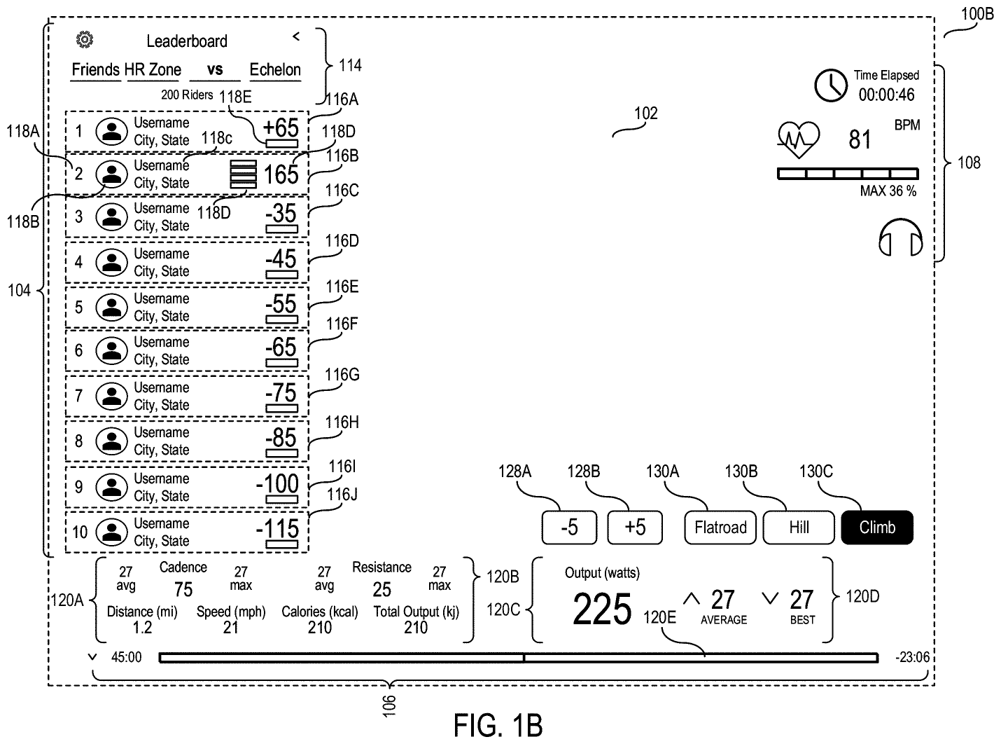

FIG. 1A is a user interface diagram of a user interface (UI) displayed on a fitness device according to some embodiments of the disclosure.

In the illustrated embodiment, a user interface 100A includes a plurality of components, including a main window 102, a leaderboard 104, a status panel 106, and a heads-up display 108. The specific arrangement of the components is not intended to be limiting.

In the illustrated embodiment, the leaderboard 104 includes a header portion that includes a title label and a plurality of tabs 114. In one embodiment, tabs 114 allow a user to toggle between various leaderboard views. In the illustrated embodiment, a versus leaderboard view is illustrated. In this view, all users performing the activity (either in real-time or in archived mode) are ranked based on their position in the activity. For example, if the activity is cycling or rowing, the users can be ranked based on their total distance cycled. Other techniques can be used to rank users, and the disclosure is not limited in that regard.

In some embodiments, leaderboard 104 is scrollable. Thus, while only ten users are listed, a user can scroll to view all users in ranked order. In some embodiments, the device implementing the user interface 100A will only load a subset of all riders for display and will issue a network request to pre-load additional riders as the user scrolls (e.g., fifty at a time). In other embodiments, the user interface 100A will pre-load all users to reduce the lag during scrolling.

In the illustrated embodiment, leaderboard 104 includes a plurality of panels 116a-116j, each panel associated with a user. Each panel may include a rank number 118a, a profile picture or icon 118b, descriptive text 118c, a relative position 118d, a current output indicator 118e, and a power meter 118f.

In the illustrated embodiment, the rank number 118a comprises an integer ranking of the users. As illustrated, users are ranked from one to the total number of users. In the illustrated embodiment, this rank number 118a comprises the sorting key when sorting users. In other embodiments, the users are ranked by a metric (e.g., distance) and then assigned a rank number 118a.

In the illustrated embodiment, the profile picture or icon 118b may comprise a user-uploaded image or a stock image, or a live image of the user as they engage in the activity. In one embodiment, icon 118b is associated with an image file transmitted to the fitness device implementing the UI. In other embodiments, the icon 118b is associated with a uniform resource locator (URL) that is dynamically downloaded when referenced in the UI. In some embodiments, the icon 118b may be augmented with badges or other indicators. In some embodiments, these badges or other indicators may comprise awards, progress indicators, or other graphics.

In the illustrated embodiment, the descriptive text 118c comprises text associated with a given user. For example, the descriptive text 118c can comprise a username, city, state, or other text data associated with the user.

In the illustrated embodiment, the relative position 118d comprises an integer value that indicates the relative position of users. In the illustrated embodiment, current user panel 116b is associated with a user using the fitness device implementing the user interface 100A. In one embodiment, the relative position 118d comprises the total output of a user. In one embodiment, the output of a user refers to a value generated based on the recorded sensor data of the fitness device. For example, an exercise bike may compute an output value based on the revolutions per minute (rpm) and the current resistance level. As another example, a rowing machine may use a stroke rate and split rate to compute an output level. Although output level is used as an example, other comparable values may be used. For example, the relative position 118d may comprise a total traveled distance during an activity, a total time performing the activity, or other value. In general, however, the relative position 118d may comprise an aggregated or total value versus an instantaneous value.

In the illustrated embodiment, panels 116a and 116c through 118j include a current output indicator 118e. In the illustrated embodiment, the current user's panel 116b does not include such an indicator. In some embodiments, however, the current user panel 116b may include such an indicator. The current output indicator 118e can comprise a visual indicator of a current output level. In one embodiment, the current output indicator 118e comprises a rectangular (or another shape) solid that changes color based on the instantaneous output of each user. In one embodiment, an instantaneous output is categorized into categories to assist in color determination. For example, an instantaneous output may be categorized as high (red), medium (orange), low (yellow), or idle (green). Thus, the current output indicator 118e changes colors based on the current output of a given user. In this manner, the current outputs of all users can be quickly understood by the current user. In some embodiments, the current output indicator 118e may further be adjusted in other ways based on the current output. For example, the width of the current output indicator 118e may be adjusted in a similar fashion. That is, the width may be adjusted to 100% (high), 75% (medium), 50% (low), or 25% (idle) of the maximum width based on the instantaneous output.

In the illustrated embodiment, the current user panel 116b does not include a current output indicator 118e. By contrast, in the illustrated embodiment, the current user panel 116b includes a power meter 118f. In the illustrated embodiment, the power meter 118f comprises a meter level indicating a user's current output. In some embodiments, the power meter 118f uses the same strategy for indicating current output. However, in the illustrated embodiment, the power meter 118f comprises a vertical stack of bars indicating instantaneous output. Continuing the previous example, in one embodiment, the lowermost bar comprises a green (idle) bar, followed upwardly by an orange bar (low), yellow bar (medium), and red bar (high). As the current user's instantaneous output changes, the appropriate bar is displayed, all bars under the appropriate bar are also displayed, and all bars above the appropriate bar are not displayed.

As discussed, leaderboard 104 comprises one example of a leaderboard. Another example of a leaderboard is further discussed herein.

FIGS. 1B, 1C, and 1D are user interface diagrams of Uls displayed on a fitness device according to some embodiments of the disclosure. Various elements bearing identical numerals as those depicted in FIG. 1A are illustrated, and the discussion of those identically numbered elements is not repeated herein.

In the UI 100B, a user is performing an activity at a first resistance level (25), as illustrated in metrics 120B. Although the illustrated UI 100B displays resistance, other metrics may be displayed based on the type of activity. For example, if the activity is indoor running on a stationary treadmill, the metrics 120B can include an incline amount. In general, the metric illustrated in metrics 120B can comprise any adjustable metric of a fitness device.

In the UI 100B, a plurality of controls 128A, 128B, 130A, 130B, 130C are depicted. In one embodiment, the controls 128A, 128B, 130A, 130B, 130C are situated above metrics 120A-120D on the UI 100B. In other embodiments, controls 128A, 128B, 130A, 130B, 130C can situated in other locations.

In one embodiment, the metrics 120A-120D can be adjustable or swappable. As illustrated in FIG. 1, an “Output (watts)” metric is currently highlighted by enlarging the metric value (“225”) and the corresponding metric values (“Average” and “Best”). In one embodiment, the highlighted metric can be replaced with an unhighlighted metric such as the “Cadence” and “Resistance” metrics illustrated in FIG. 1. Specifically, in one embodiment, a user can interact with an unhighlighted metric by, for example, double tapping, long pressing, clicking, or some other form of interaction. In response, the unhighlighted metric the user interacts with can be swapped with the currently highlighted metric. For example, in FIG. 1, if a user interacts with the “Cadence” metric, the “Cadence” metric and “Output” metric can swap with the values of the “Output” metric being reduced in size and the values oef the “Cadence” metric being increased in size.

In one embodiment, the unhighlighted metric interacted with by the user and the highlighted metric can be swapped directly. In other embodiments, a rotating swap can be performed. For example, if the user interacts with the “Cadence” metric, the “Cadence” metric can replace the “Output” metric, the “Output” metric can replace the “Resistance” metric, and the “Resistance” metric can replace the previous “Cadence” metric. In this manner, the three metrics “rotate” counterclockwise when interacted with. In one embodiment, this rotating can only be performed when the left-most metric is selected. If the user selects the closest metric, a direct swap may be performed instead.

In one embodiment, animations can be performed during the above swap. For example, the metrics can physically be moved on screen, visualizing the swap. By contrast, in other embodiments, the metrics can be made invisible and re-appear in the final locations. In one embodiment, the opacity of the metrics can be reduced while the metrics are moving and restored to a non-transparent or non-translucent state when reaching their final locations. In one embodiment, the metrics change size and can be scaled (up or down) proportionate to the distance to the final location.

In one embodiment, the above swapping can be done by a user of the fitness equipment. However, in other embodiments, the above swapping can be performed by an instructor or other remote user. In this embodiment, the remote user can transmit an instruction that causes the user interface to update in the above-described manner. For example, a class instructor can cause a user's user interface to swap the “Output” metric with the “Cadence” metric to focus on cadence during a workout.

The controls 128A, 128B, 130A, 130B, 130C can comprise any user-selectable component capable of being displayed in the UI 100B. For example, the controls 128A, 128B, 130A, 130B, 130C can comprise a button or other similar UI element amenable to receiving a touch event. In one embodiment, controls 128A, 128B comprise selectable individually selectable controls. That is, controls 128A, 128B can be selected independently, and each selection causes an action to occur. In one embodiment, the controls 128A, 128B can modify the level of resistance or another metric level. As illustrated, control 128A can cause the value of the metric level to be increased by five when selected, whereas control 128B can cause the value of the metric level to be decreased by five when selected. In some embodiments, the value of the metric includes a minimum and maximum value. In some embodiments, when the metric level is equal to the minimum value, the control 128A may be disabled. Similarly, when the metric level is equal to the maximum value, the control 128B may be disabled. In one embodiment, each control 128A, 128B is attached to an event handler to modify the value of the metric (e.g., resistance) level. Certainly, more controls meeting the above functionality can be displayed. Thus, controls allowing for ±1, ±10, ±20, etc., can be provided in addition to or alternatively to the controls 128A, 128B. In some embodiments, the specific numeric values of controls 128A, 128B can be customized by the user (similar to controls 130A, 130B, 130C, discussed next).

In the UI 100B, the controls 130A, 130B, 130C can comprise selectable components of a radio group. As used herein, a radio group refers to a set of controls that select mutually exclusive options. Thus, as illustrated, one control (130C) is visual distinguished from the others (130A, 130B) when it is selected. Further, after a user selects a control (130C), the control (130C) remains active until the underlying value is changed. For example, control (130C) may remain active until the user selects another control 128A, 128B, 130A, 130B.

In the illustrated embodiment, the controls 130A, 130B, 130C are each associated with a preset metric level of the metric level. For example, each of the 130A, 130B, 130C can be associated with a user-configurable resistance level. For example, control 130A can be associated with a +5 resistance level, and control 130B can be associated with a +20 resistance level, and control 130C can be associated with a +40 resistance level. As illustrated, each control 130A, 130B, 130C can be associated with a static (or customizable) descriptive label (“Flatroad,” “Hill,” “Climb”) to enable easy understanding of the presets. In one embodiment, these labels are customizable. Further, in one embodiment, the preset metric levels can be customized. Thus, a user can change the numeric value of each of the controls 130A, 130B, 130C. When a user selects one of the controls 130A, 130B, 130C, the corresponding metric level can be changed to the corresponding preset metric level.

In one embodiment, when a user selects one of the controls 128A, 128B, 130A, 130B, 130C, the corresponding metric (e.g., resistance) level can be changed immediately. In another embodiment, the corresponding metric (e.g., resistance) level can be changed gradually according to a preset decay factor. For example, the metric level can be increased linearly over a preconfigured duration (e.g., one second).

No limitation is placed on when a user can select one of the controls 128A, 128B, 130A, 130B, 130C. For example, the user can arbitrarily select one of the controls 128A, 128B, 130A, 130B, 130C to change the metric level. As another example, the user can select one of the controls in response to a visual or audio prompt from a class displayed in main window 102.

In some embodiments, the controls can be pressure-sensitive. Thus, for example, while controls 128A, 128B can default to a ±5 increase in the metric level, a “long push” (e.g., durationally or based on pressure) can be used to increase the metric level by more than ±5. In some embodiments, a numeric value displayed in controls 128A, 128B can be dynamically changed as the user pushes the controls 128A, 128B.

In some embodiments, the controls can have different visual appearances. For example, as illustrated in FIG. 1C, radio controls 132A, 132B, 132C can comprise both textual content (“Flatroad,” “Hill,” “Climb”) as well as corresponding icons. In the illustrated embodiment, the icons are situated left of the textual content; however, the illustrated embodiment is not limited as such. In a similar manner, in some embodiments, the controls can omit textual content and include only icons as illustrated in controls 134A, 134B, 134C of FIG. 1D. In some embodiments, an icon-only approach, as illustrated in FIG. 1D, may be used to maximize screen real estate. In some embodiments, all three approaches in FIGS. 1B, 1C, and 1D may be used. For example, a user may select switch visual appearance to utilize on the UI. In some embodiments, the controls may vary in content when selected. For example, in an unselected state, controls 134A, 134B, 134C may be displayed. During a selection operation, the selected control may add textual content such as that depicted in 132A, 132B, 132C. In some embodiments, a selected control can differ in content from that of unselected controls. For example, a selected control (e.g., 132C) can include an icon and text content, while unselected controls can include only icon content (e.g., 134A, 134B).

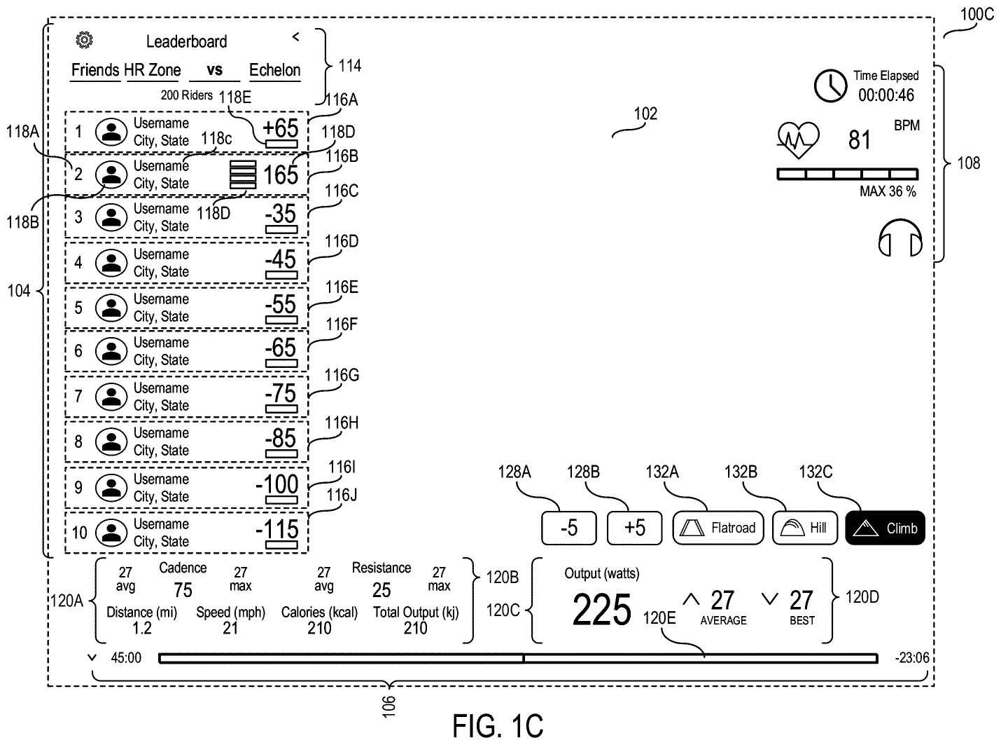

FIG. 2 is a user interface diagram of a leaderboard 200 displayed on a fitness device according to some embodiments of the disclosure. In some embodiments, the leaderboard 200 may display in lieu of leaderboard 104 of FIG. 1A.

In the illustrated embodiment, a leaderboard 200 includes a plurality of panels 202a-202e. The specific number of panels is not limiting. As with leaderboard 104, the leaderboard 200 ranks the users associated with panels 202a-202e based on a comparable metric (e.g., total output). A given panel 202a is given an icon 204a and includes descriptive text 206a. Details of these elements are similar to rank number 118a and descriptive text 118c, respectively, and those details are not repeated herein. In the illustrated embodiment, a given panel 202a includes an icon 204a. In one embodiment, icon 204a comprises a user profile picture or a stock image. In other embodiments, icon 204a comprises a ranking image (e.g., an image of the user's integer rank). In some embodiments, an integer ranking may be overlaid on top of an image of the respective user.

In contrast to user interface 100A, leaderboard 200 includes variable spacing 208a-208d between panels 202a-202e. In one embodiment, the spacing 208a-208d is determine based on the difference between outputs of adjacent panels 202a-202e. For example, the output levels associated with panels 202a and 202b may comprise 180 and 160, respectively; thus, the difference is 20. In one embodiment, the spacing 208a may be computed as 20 pixels. Similarly, the output levels of panels 202b and 202c may comprise 160 and 100. Thus, the spacing 208b may be computed as 60 pixels.

In some embodiments, the spacing 208a-208d may be adjusted based on the number of panels and the available space. For example, if the distance between panels 202a and 202b is too large, the leaderboard 200 may only display two or even one panel. In some embodiments, the leaderboard 200 will attempt to display a constant number of users (e.g., five). Thus, the leaderboard 200 can evenly space the panels 202a-202e first and determine the available spacing 208a-208d. Next, the leaderboard 200 can represent the distances between panels 202a-202e as a percentage of the available space. For example, if each panel is 50 pixels high, 250 pixels are needed to render the panels. If the total height of the leader board is 450 pixels, then 200 pixels of spacing is available. If the difference in total output between the riders is equal (e.g., 50), the spacing 208a-208d is evenly divided among the 200 pixels. Thus, each spacing 208a-208d is set to 50 pixels. However, if the distance between panels 202a and 202b increases to 100 while the distances between the other panels remain 50, the leaderboard 200 may adjust the spacing by computing the distance percentage between panels 202a and 202b (e.g., 100/(100+50+50+50)=40%) as well as the percentages for the remaining spacings (e.g., 50/(100+50+50+50)=20%). The leaderboard 200 will then use these percentages to compute the spacings 208a, 208b, 208c, and 208d as 80, 40, 40, and 40 pixels, respectively. In this manner, a fixed number of panels 202a-202e can be displayed at any moment.

Returning to FIG. 1A, the user interface 100A can further include a status panel 106. In the illustrated embodiment, status panel 106 includes various metrics 120A-120D measured for a current user's participation in an activity. For example, as illustrated, the status panel 106 includes a user's cadence, resistance level, total output (in watts). As illustrated, status panel 106 may display an instantaneous value for each of these metrics as well as average and maximum recorded values for a given activity. In some embodiments, the status panel 106 further includes data such as distance, speed, calories burned, and a progress bar 120e indicating the user's progress in performing an activity.

The user interface 100A may further include a heads-up display 108. In the illustrated embodiment, the heads-up display 108 may include various details of the activity such as the time elapsed, a heart rate, heart rate training zones, etc.

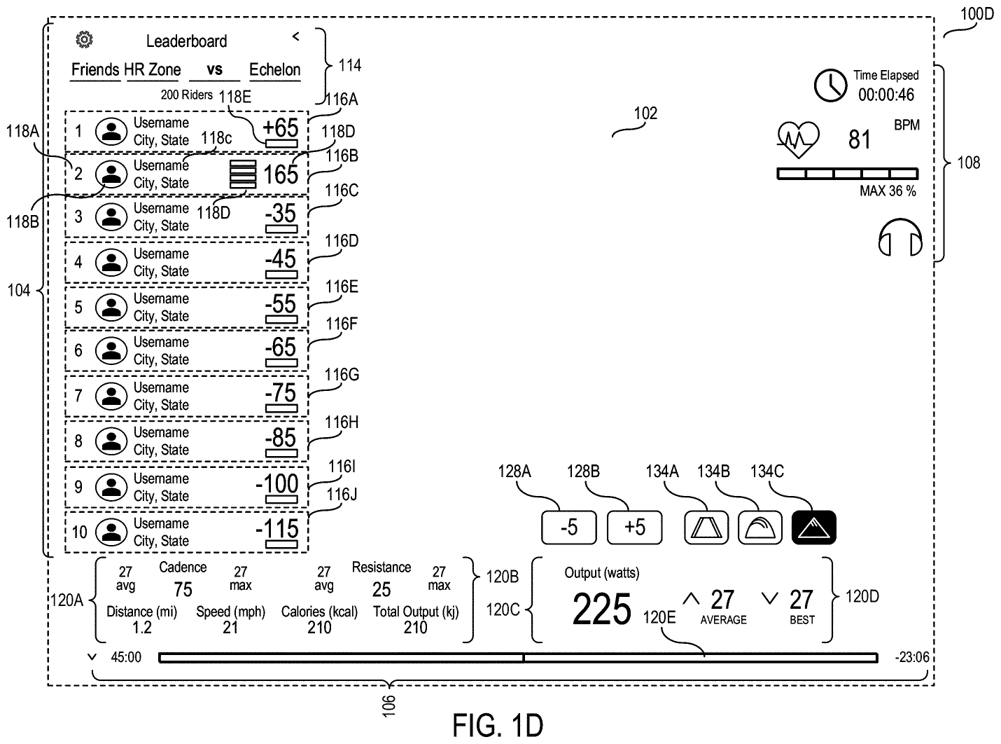

In the illustrated embodiment, the user interface 100A includes a main window 102. In the illustrated embodiment, main window 102 includes a curved graph 110 having a plurality of icons 112a-112c situated thereon. The detail of graph 110 is expanded upon in FIG. 3.

In the various embodiments described above, a user may be able to minimize, hide, collapse, or otherwise manipulate the various sections 102, 104, 106, and 108 of the user interface 100A. For example, the user may be able to swipe leaderboard 104 to the left (via a capacitive touchscreen) to hide the leaderboard 104. The user may then be able to swipe right from the edge of the screen to display the leaderboard 104 again. In another embodiment, icons may be presented on sections 102, 104, 106, and 108 to hide the sections. In these embodiments, sections 102, 104, 106, and 108 may be overlaid on top of a full-screen video stream. In some embodiments, sections 102, 104, 106, and 108 may be opaque, while in others, some or all of the sections 102, 104, 106, and 108 may be translucent.

In the illustrated embodiment, a single curve 110 is illustrated having multiple icons 112a-112c situated thereon. However, in other embodiments, multiple curves may be presented, one for each of the icons 112a-112c. In this embodiment, the curves for each of the icons 112a-112c may be identical and the positioning of each of the icons 112a-112c may be performed in a similar manner as described. However, in this embodiment, each graph only shows a single icon of the icons 112a-112c. In one embodiment, the graphs may be aligned along the bottom of the main window 102.

FIG. 3 is a user interface diagram of the main window displayed on a fitness device according to some embodiments of the disclosure.

In the illustrated embodiment, the curved graph 110 comprises a sinusoidal curve, although other types of curves can be used, such as parabolic curves, etc. In the illustrated embodiment, a plurality of icons 112a-112c is positioned along the curve 110. Each icon 112a is associated with a corresponding center 126a-126c. In some embodiments, the centers comprise a point equidistant from every point on the perimeter of icons 112a-112c. In other embodiments, other points may be used. In the illustrated embodiment, the centers 126a-126c are situated along the curve 110.

In the illustrated embodiment, a y-axis 124 and x-axis 122 are illustrated, with an original at the intersection of the y-axis 124 and x-axis 122. The y-axis 124 and x-axis 122 are illustrated solely for explanation purposes and do not appear in the visualization. In the illustrated embodiment, the centers 126a-126c of each icon 112a-112c are associated with a point relative to the origin of the y-axis 124 and x-axis 122. Thus, icon 112a is associated with a center 126a having coordinates of (p₁, h₁). In the illustrated embodiment, the position points (p₁, p₂, p₃) are determined based on the ranking of the users, as discussed with respect to icon 118b. Thus, in a racing scenario, the user associated with icon 112c leads the user associated with icon 112b, and the user associated with icon 112b leads the user associated with icon 112a (i.e., p₃>p₂>p₁).

In the illustrated embodiment, the height (i.e., distance from x-axis 122) is determined based on the instantaneous output of the users associated with icons 112a-112c. For example, the current energy output (e.g., in kilojoules) may be used to compute the heights h₁, h₂, h₃. In one embodiment, the heights are normalized to fit within a maximum height of the main window 102.

In one embodiment, the centers 126a-126c are used to fit the formula of the curve 110. In one embodiment, a sinusoidal model is used to generate a formulaic representation of the curve 110 which is then used to render the points along the curve, including centers 126a-126c used to place the icons 112a-112c. In other embodiments, a default curve may be used first and the coordinates of centers 126a-126c may be used to adjust coordinates of the default curve. The curve 110 may then be adjusted to match these coordinates, resulting in the final curve. Notably, the curve may have a varying amplitude and frequency.

In one embodiment, the coordinates of the centers 126a-126c change through the performance of an activity. Thus, the heights and positions of the centers 126a-126c may be moving throughout the activity. In this scenario, the display of the curve 110 is redrawn each time a center 126a-126c change position, thus moving along with the change in instantaneous output and positions.

In general, curve 110 may be represented by, for example, the formula y(t)=h(t) sin (p(t)), where h(t) is a function outputting the time-varying height of the centers 126a-126c and p(t) represents the time-varying positions of the centers 126a-126c.

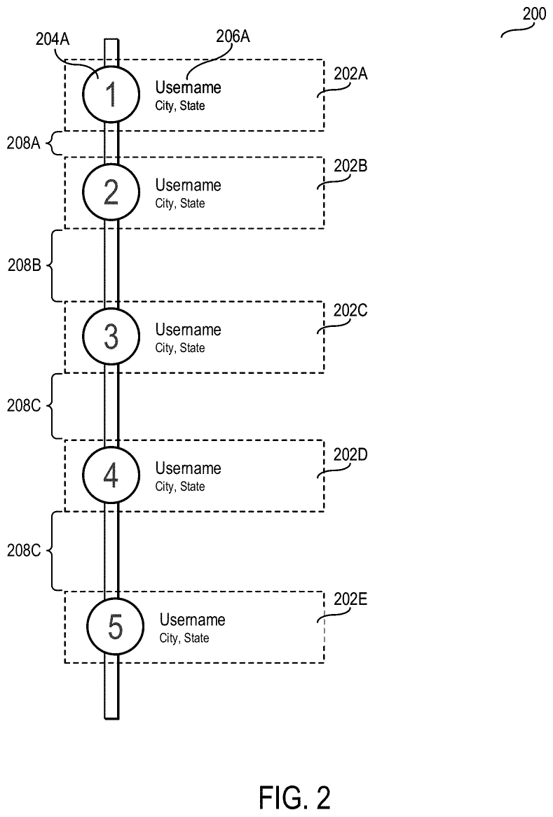

FIG. 4 is a flow diagram illustrating a method for generating a visualization on a fitness device according to some embodiments of the disclosure.

In step 402, method 400 receives leaderboard data.

In one embodiment, step 402 includes receiving, at a fitness device over a network, activity data from a server, the activity data comprising data reported by other fitness devices during participation in an activity. In one embodiment, step 402 includes extracting user data, position data, and output data from the activity or leaderboard data.

In the illustrated embodiment, while a user is participating in an activity (e.g., cycling class, rowing class, running class, etc.) the fitness device periodically receives leaderboard data from a remote computing device (e.g., server). As discussed in the previous figures, this leaderboard data comprises a ranked list of users also participating in the activity, either in real- or near-real-time or from an archived class. For example, the data may include user data, a current total output, and an instantaneous output:

```
[
  {“user”: 1, “output”: 250, “instantaneous”: “50”},
  {“user”: 3, “output”: 220, “instantaneous”: “75”},
  {“user”: 2, “output”: 200, “instantaneous”: “90”},
]
```

As illustrated, the data may be pre-sorted by, for example, a total output. This sorting corresponds to the rankings described above. In some embodiments, the data may be unsorted and sorted by the fitness device.

In some embodiments, the leaderboard data is received for a live class. In this embodiment, the leaderboard data comprises data associated with users participating in the live activity at the current time. In other embodiments, the leaderboard data is received for an archived activity. In these embodiments, the leaderboard data comprises data associated with users participating in an archived activity at a given moment of playback of the archived activity.

In step 404, method 400 computes a visualization window for displaying leaderboard data.

In one embodiment, the leaderboard data includes data of the current user of the fitness device executing the method. For example, the fitness device may transmit its instantaneous and total output to a central server and may receive, in response, updated leaderboard data that includes their uploaded data and all other data associated with users performing (or that have performed) the activity. In another embodiment, the leaderboard may not include the current user's data but rather the current user's data may be recorded locally. In these embodiments, method 400 may include inserting the local data into the received leaderboard data in the appropriate ordered location.

In either scenario, the visualization window refers to a subset of the data in the leaderboard data. For example, the visualization window may comprise five total users in the leaderboard data. In one embodiment, method 400 centers the visualization window around a current user and selects an equal number of users that are ranked higher and lower than the current user. For example, if the visualization window comprises five users, method 400 may select the two users ahead of the current user and the two users behind the current user. If the current user is the first user in the ordering, method 400 may select four users after the current user and if the current user is the last user, method 400 may select four users ahead of the current user.

In step 406, method 400 generates a curve based on the leaderboard data in the visualization window.

In one embodiment, method 400 generates a curve by fitting a curve to the data selected for the visualization window. As discussed, in one embodiment, method 400 may compute coordinates for each piece of user data. In one embodiment, method 400 selects the middle user in the visualization window which, as an example, may be the current user.

In one embodiment, method 400 generates a curve by generating a base curve. In one embodiment, method 400 generates a base sinusoidal curve. In one embodiment, the frequency and amplitude of the base curve are determined based on the available area to render the curve. For example, method 400 may select a frequency such that two periods are visible in the available area. Method 400 may further center the curve vertically such that the amplitude is one-half of the height of the available area. Other determinations may be made. After generating an initial curve, method 400 plots the leaderboard in the visualization window. As discussed, in one embodiment, the data in the leaderboard may be used to generate an x and y position for each data point. Method 400 superimposes these points on the base curve and then adjusts the curve based on the plotted points. For example, method 400 may select each point and identify a point on the base curve having the same x position (but potentially different y position). Method 400 will then adjust the y position of that point on the curve to match the corresponding leaderboard data point. After adjusting the points, method 400 smooths all remining points of the curve to render a final continuous curve.

In an alternative embodiment, method 400 may use the leaderboard data as an initial set of points and fit a curve to those points. As discussed, the metrics in the leaderboard data may be used as x and y coordinates of the initial set of points. Using this set, method 400 may fit a curve (e.g., sinusoidal, n-degree polynomial) to the initial set of points.

In step 408, method 400 renders the curve. In one embodiment, method 400 comprises displaying the curve in a main window of UI. In one embodiment, method 400 may scale the rendering of the curve to fit within a preconfigured area of the main window.

In step 410, method 400 determines if the activity is over. If not, method 400 continuously re-executes steps 402, 404, 406, and 408. Alternatively, if method 400 determines if the activity is over, method 400 ends.

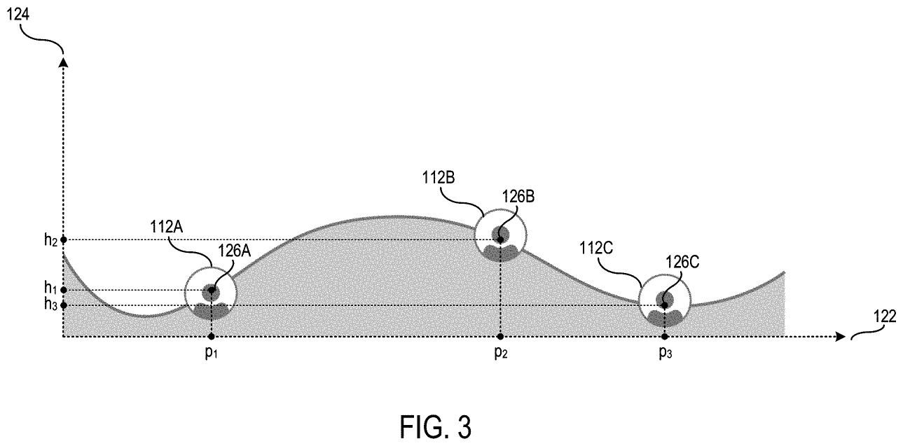

FIG. 5 is a flow diagram illustrating a method (500) for propagating activity data according to some embodiments of the disclosure. In some embodiments, the method (500) can be executed by a single server. In other embodiments, the method (500) may be executed by a cloud platform comprising a plurality of physical servers and virtualized servers or containers.

In step 502, method 500 receives a join message. In one embodiment, method 500 receives the join message from a given fitness device. In one embodiment, the join message includes an activity identifier or similar mechanism to identify an activity to join. For example, if the activity comprises a spin class, the identifier may comprise a class identifier. In one embodiment, the join message further includes information identifying the requesting user and/or the fitness device. For example, the join message may further include a user identifier or account name. In some embodiments, the join message is transmitted over a socket-based connection such as a WebSocket connection.

In step 504, method 500 determines if the activity is queued. In one embodiment, in response to a join message, method 500 will determine if a data structure representing the activity is present in a cache data store. If so, method 500 will bypass step 506. Alternatively, if method 500 determines that the activity is not queued, it queues the activity in step 506.

In one embodiment, a given activity is associated with set of participants and each participant is associated with metrics representing their performance of that activity. In the illustrated embodiment, the cache data store maintains, for each active activity, an in-cache representation of this leaderboard data.

If the join message is the first user of a live class, no such data will be cached. Thus, method 500 will create an initial data structure as part of queuing the activity in step 506. In one embodiment, creating the initial data structure may comprise creating a representation of the user that issued the join message in the leaderboard data structure and writing the leaderboard data structure to the cache. Alternatively, if the join message is received from a subsequent user, method 500 will determine that the activity is queued and will simply add the user to the leaderboard. For archived activity, the process is similar. In some embodiments, when a user requests an archived activity, method 500 will proceed to step 506 to create an initial leaderboard. Method 500 may then query a database to identify past performances of the activity and populate the leaderboard with data from those past performances. In some embodiments, these past performances may comprise other users that have completed the activity in the past. Alternatively, or in conjunction with the foregoing, the other users may include the same user's past performances (i.e., the current user can compete against themselves).

Once the activity is properly queued, method 500 proceeds to start the activity in step 508. For live activities, method 500 may comprise awaiting a global start timestamp. Once the current time is equal to the global start timestamp, method 500 may stream live video content. Concurrently, method 500 will proceed to step 510 to begin collecting statistics from live participants. For archived classes, method 500 may immediately begin streaming archived video content and proceed to step 510 to begin collecting statistics from the current user. Concurrently, for archived classes, method 500 may continuously update the cached leaderboard data based on historical performance data.

In step 510, method 500 receives activity statistics and UI state data from a fitness device. In one embodiment, statistics refer to measurable performance of the activity. For example, the measurable performance may comprise the current output of a spin bike or rowing machine. In some embodiments, the statistics maybe timestamped. In some embodiments, the timestamping is relative to a streaming or archived video stream. In one embodiment, UI state data may comprise the displayed users on a given fitness device at a given time. For example, method 500 may receive the visualization window discussed previously. In other embodiments, the UI state data may comprise a request for a new set of leaderboard data for a current time. In some embodiments, the statistics and UI state data are combined in a single message. However, in some embodiments, the messages are separate. In a separated example, method 500 may receive statistics at a more rapid (i.e., near real-time) frequency than UI state data requests.

In step 512, method 500 writes the stats to the canonical activity data stores. In one embodiment, method 500 persists the received states to a database or similar long-term storage. These persisted data points can then be used for archived classes. In some embodiments, method 500 may further write the stats to the cache.

In step 514, method 500 returns updated activity. In one embodiment, method 500 returns the leaderboard data (discussed previously). In one embodiment, method 500 returns the leaderboard data in response to a UI state data request. In some embodiments, this request is separate from an update of statistics, as discussed. An example of the format of the returned leaderboard data has been described previously and is not repeated herein. In one embodiment, method 500 reads the current leaderboard data for the identified class by querying the cache and retrieving the most recent leaderboard data. In some embodiments, method 500 may further sort the leaderboard data prior to returning it.

In step 516, method 500 determines if a leave message was received. In one embodiment, a fitness device may transmit a leave message upon exiting an activity. If the user remains in an activity, method 500 continuously executes steps 510, 512, and 514 until the user leaves the activity or the activity ends.

In step 518, after the user leaves the activity, method 500 persists the cached data for the user in a persistent data store. In one embodiment, method 500 may further remove the user data from the cache data store, thus preventing the user from being displayed on other leaderboards. By persisting the data, the user data can be re-used during archived classes.

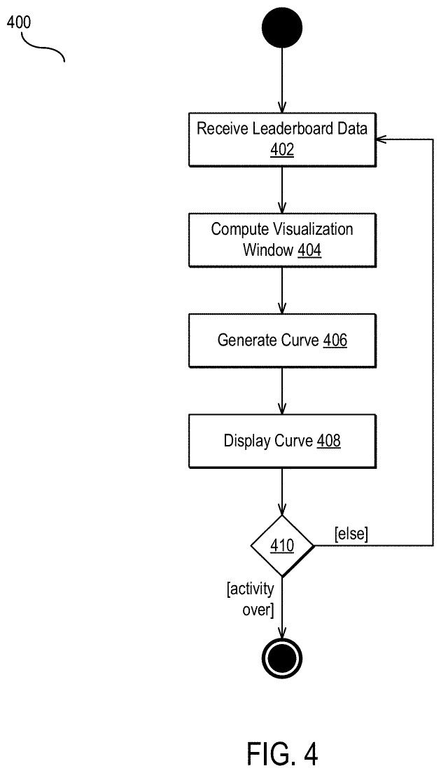

FIG. 6 is a block diagram of a system 600 for distributing and visualizing leaderboard data according to some embodiments of the disclosure.

In the illustrated embodiment, a system 600 includes plurality of fitness devices 602 are communicatively coupled to a router 604. In the illustrated embodiment, the fitness devices 602 may comprise any equipment configured to monitor activity performance of a user. Examples of fitness devices 602 include, but are not limited to, spin bikes, stationary bikes, rowing machines, fitness mirrors, weightlifting apparatuses, and other types of fitness-related equipment. In general, any device capable of capturing an activity metric may be used. Examples of activity metrics include power output or resistance for spin bikes, stroke rate and split time for rowing machines, calories burned, heartrate, etc. In some embodiments, fitness devices 602 may comprise computing devices not specifically designed for fitness. For example, a smart watch may comprise a fitness device and may record a current lap number (or distance) while a user is swimming. The aforementioned visualization can be displayed on the watch and updated based on the measured laps (or distance).

In the illustrated embodiment, the fitness devices 602 transmit and receive data via router 604. In one embodiment, the router 604 comprises a message broker or similar application/hardware that receives messages and routes messages to the appropriate server in the application servers 606. In one embodiment, the router 604 may comprise a publish-subscribe broker, although other routers may be used. As discussed in connection with FIG. 5, the router 604 receives messages such as join, stats, UI state, and leave messages.

In the illustrated embodiment, the system 600 includes a plurality of application servers 606. In one embodiment, these application servers 606 comprise dedicated hardware devices. However, in other embodiments, the application servers 606 comprise virtualized servers or functions. For example, each of the application servers 606 may comprise a software container or other type of virtualized application that may be deployed on various hardware. In some embodiments, each server in the application servers 606 implements a single function. In some embodiments, multiple application servers 606 may implement a single function. In some embodiments, the application servers 606 perform some of all of the steps described in FIG. 5, which are not repeated in detail herein.

The system 600 includes a cache 608 communicatively coupled to the application servers 606. The cache 608 may comprise an in-memory data store such as Redis or similar type storage mechanisms. In the illustrated embodiment, the cache 608 stores data related to live activities or to archived activities that are being replayed. In one embodiment, the cache 608 stores a leaderboard for each activity. In one embodiment, the cache 608 includes internal sorting functionality (e.g., via a Redis SortedSet) to enable rapid sorting of large data volumes. In the illustrated embodiment, the cache 608 can further allow arbitrary reads and writes to data structures stored within.

In the illustrated embodiment, the system 600 further includes at least one database 610. In the illustrated embodiment, the least one database 610 may comprise a database cluster. In other embodiments, the database 610 may comprise a big data storage platform. In general, the least one database 610 is configured to store all recorded data for all activities. This includes all data recorded during a live activity as well as all data generated during a replay of an archived activity.

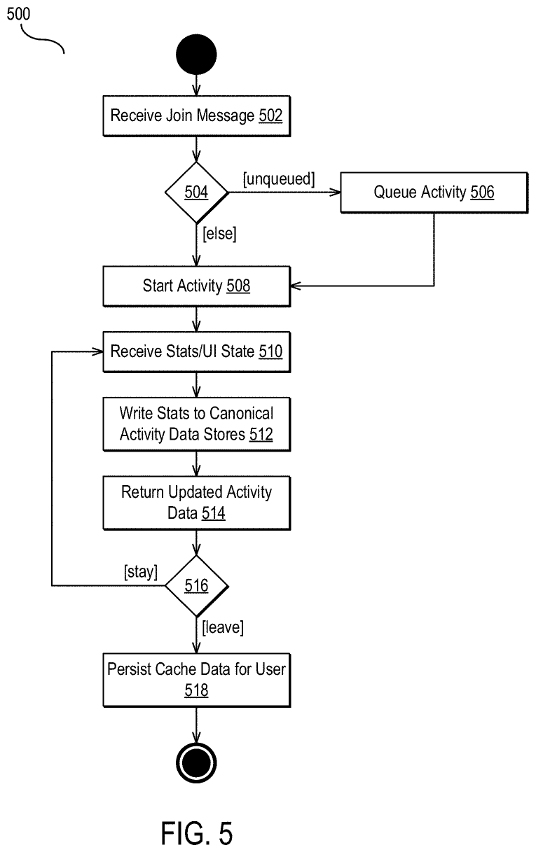

FIG. 7 is a block diagram illustrating a computing device showing an example of a client or server device used in the various embodiments of the disclosure.

The computing device 700 may include more or fewer components than those shown in FIG. 7, depending on the deployment or usage of the device 700. For example, a server computing device, such as a rack-mounted server, may not include an audio interface 752, display 754, keypad 756, illuminator 758, haptic interface 762, Global Positioning Service (GPS) receiver 764, or cameras/sensor 766. Some devices may include additional components not shown, such as graphics processing unit (GPU) devices, cryptographic co-processors, artificial intelligence (AI) accelerators, or other peripheral devices.

As shown in the figure, the device 700 includes a central processing unit (CPU) 722 in communication with a mass memory 730 via a bus 724. The computing device 700 also includes one or more network interfaces 750, an audio interface 752, a display 754, a keypad 756, an illuminator 758, an input/output interface 760, a haptic interface 762, an optional GPS receiver 764 and a camera(s) or other optical, thermal, or electromagnetic sensors 766. Device 700 can include one camera/sensor 766 or a plurality of cameras/sensors 766. The positioning of the camera(s)/sensor(s) 766 on the device 700 can change per device 700 model, per device 700 capabilities, and the like, or some combination thereof. In some embodiments, the sensors 766 may comprise fitness-specific sensors such as a revolutions per minute sensor on a spin bike or an ergometer on a rowing machine.

In some embodiments, the CPU 722 may comprise a general-purpose CPU. The CPU 722 may comprise a single-core or multiple-core CPU. The CPU 722 may comprise a system-on-a-chip (SoC) or a similar embedded system. In some embodiments, a GPU may be used in place of, or in combination with, a CPU 722. Mass memory 730 may comprise a dynamic random-access memory (DRAM) device, a static random-access memory device (SRAM), or a Flash (e.g., NAND Flash memory device). In some embodiments, mass memory 730 may comprise a combination of such memory types. In one embodiment, the bus 724 may comprise a Peripheral Component Interconnect Express (PCIe) bus. In some embodiments, the bus 724 may comprise multiple busses instead of a single bus.

Mass memory 730 illustrates another example of computer storage media for the storage of information such as computer-readable instructions, data structures, program modules, or other data. Mass memory 730 stores a basic input/output system (“BIOS”) 740 for controlling the low-level operation of the computing device 700. The mass memory also stores an operating system 741 for controlling the operation of the computing device 700

Applications 742 may include computer-executable instructions which, when executed by the computing device 700, perform any of the methods (or portions of the methods) described previously in the description of the preceding Figures. In some embodiments, the software or programs implementing the method embodiments can be read from a hard disk drive (not illustrated and temporarily stored in RAM 732 by CPU 722. CPU 722 may then read the software or data from RAM 732, process them, and store them to RAM 732 again.

The computing device 700 may optionally communicate with a base station (not shown) or directly with another computing device. Network interface 750 is sometimes known as a transceiver, transceiving device, or network interface card (NIC).

The audio interface 752 produces and receives audio signals such as the sound of a human voice. For example, the audio interface 752 may be coupled to a speaker and microphone (not shown) to enable telecommunication with others or generate an audio acknowledgment for some action. Display 754 may be a liquid crystal display (LCD), gas plasma, light-emitting diode (LED), or any other type of display used with a computing device. Display 754 may also include a touch-sensitive screen arranged to receive input from an object such as a stylus or a digit from a human hand.

Keypad 756 may comprise any input device arranged to receive input from a user. Illuminator 758 may provide a status indication or provide light.

The computing device 700 also comprises an input/output interface 760 for communicating with external devices, using communication technologies, such as USB, infrared, Bluetooth™, or the like. The haptic interface 762 provides tactile feedback to a user of the client device.

The optional GPS receiver 764 can determine the physical coordinates of the computing device 700 on the surface of the Earth, which typically outputs a location as latitude and longitude values. GPS receiver 764 can also employ other geo-positioning mechanisms, including, but not limited to, triangulation, assisted GPS (AGPS), E-OTD, CI, SAT, ETA, BSS, or the like, to further determine the physical location of the computing device 700 on the surface of the Earth. In one embodiment, however, the computing device 700 may communicate through other components, provide other information that may be employed to determine a physical location of the device, including, for example, a MAC address, IP address, or the like.

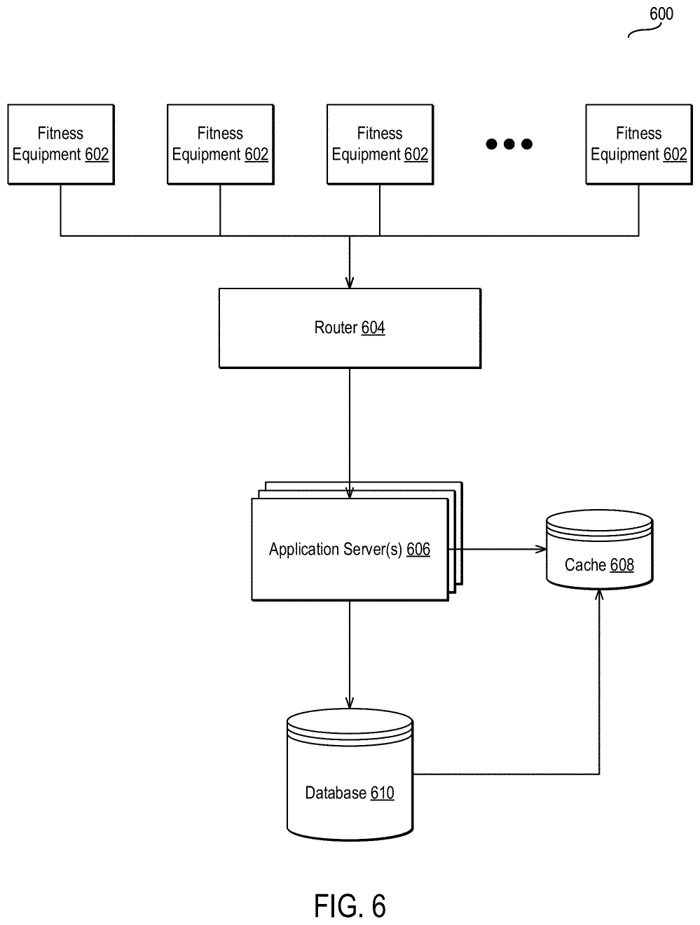

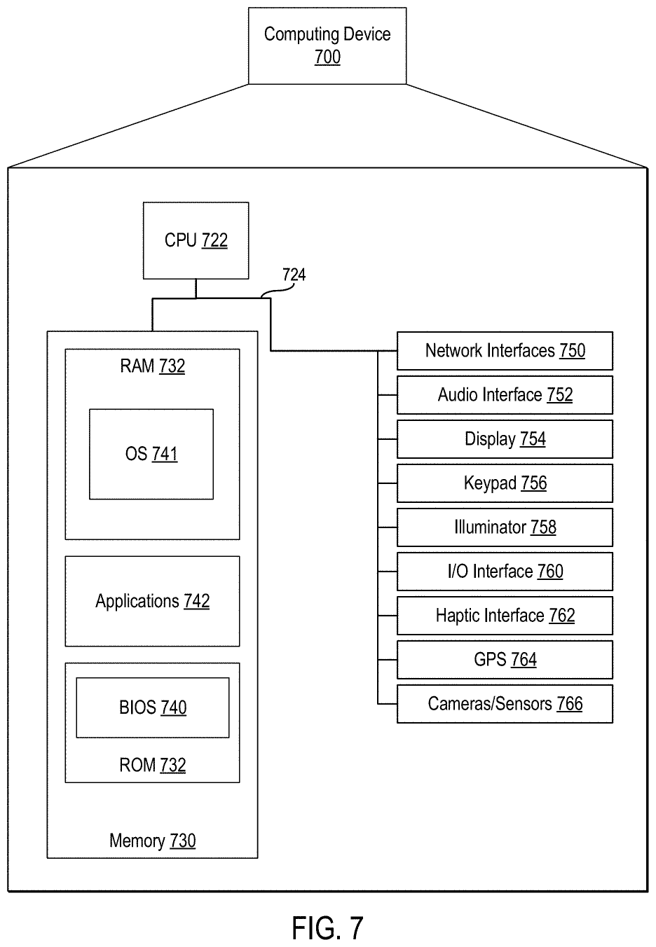

FIG. 8A is a flow diagram illustrating a method for capturing and recording a user image during an activity according to some embodiments of the disclosure.

In step 802, method 800A can comprise detecting the end of an activity. In one embodiment, as previously discussed, the end of an activity can comprise the end of a fitness class (e.g., spin class, cycling class, rowing class, workout class, etc.) or another session of predefined length. Alternatively, or in conjunction with the foregoing, the end of an activity can comprise a user leaving an activity. FIGS. 4 and 5 provide further detail on ending an activity, and that description is not repeated herein.

In step 804, method 800A can comprise recording multimedia content.

In one embodiment, multimedia content can comprise image, video, or audio content. In one embodiment, method 800A comprises operating a camera on a tablet device communicatively coupled to a fitness device. For example, a spin or exercise bike can include a tablet device mounted above handlebars and including a front-facing camera directed at the user while the user is situated on the bike. Alternatively, or in conjunction with the foregoing, a user can utilize a mobile phone or tablet that is not physically connected to the fitness device to record multimedia content. For example, a user may use his or her personal mobile phone or tablet to record multimedia content. In either scenario, the multimedia content can be transmitted to a remote server such as application servers 606.

In another embodiment, step 804 can comprise streaming multimedia content to the application servers while a user is performing an activity. In these embodiments, a remote process running on the application servers can classify image frames and/or audio of the user to detect an image or set of images to use as the multimedia content. For example, the application servers may use a machine learning (ML) model to classify image frames by emotion or expression (e.g., smiling, happy, etc.) and choose an image or set of images to present to the user for their selection. In such an embodiment, a user need not be prompted to record an image, and the image can be captured hands-free while the user is performing the activity.

In step 806, method 800A can comprise associating the multimedia content with activity statistics. In one embodiment, the activity statistics comprise statistics regarding the user's performance of an activity as described in the previous figures. As one example, activity statistics can comprise a distance traveled, a total power output, average speed, maximum speed, etc. Generally, and data recorded by a fitness device can be used as activity statistics. In some embodiments, the activity statistics include multiple such metrics packaged in a single data structure and associated with a given activity (e.g., class). Method 800A can associate multimedia content with activity data in multiple ways.

In one embodiment, if a tablet communicatively coupled to the fitness device records the multimedia content, method 800A can immediately associate the multimedia content with the activity statistics for the activity. As an example, as the user stops an activity in step 802, method 800A generates an object representing the statistics of the user during the activity. For example, upon completing a spin class, method 800A can create an object that includes the distance traveled, average power output, etc. Next, method 800A can display a prompt requesting that the user record multimedia content. For example, method 800A can display a prompt asking the user whether or not they would like to “take a selfie” now that the activity has been completed. Certainly, a user can decline the option. In that scenario, method 800A can upload the statistics without multimedia content as described previously. If, however, the user opts to record multimedia content, method 800A will record multimedia content using, for example, a microphone and/or camera installed on the tablet. Next, method 800A can combine the statistics and the multimedia content into a single message and transmit this single message to the application servers 606. Thus, in some embodiments, method 800A comprises associating statistics and multimedia content on the fitness device.

In another embodiment, the statistics may be streamed to the application servers and processed in a batch mode upon completion of the activity. In such a scenario, method 800A may not transmit a single object describing the activity statistics. Rather, the application servers can be configured to generate the activity statistics based on the streamed data. In this scenario, method 800A can capture the multimedia content (subject to the approval of the user, as previously discussed) and upload the multimedia content by itself to the application servers. In one embodiment, method 800A can transmit only the multimedia content, and the application servers can determine a corresponding activity based on a receipt time of the multimedia content. For example, upon receiving multimedia content at a given time, the application servers can find the closest completed activity that precedes receipt of the multimedia content. The application servers can then associate the multimedia content with the most recent completed activity statistics.

In another embodiment, the multimedia content can be recorded by a device other than the one that records the activity statistics. In such an embodiment, method 800A can proceed similar to the foregoing description. However, in such an embodiment, a user may log in to an account first on their external device such that the multimedia content can be associated with the user prior to uploading to the application servers. In some embodiments, a mobile application on the user's device can receive the activity statistics first, and the user can record multimedia content within the mobile application. In such a scenario, method 800A can proceed according to the first embodiment described above (wherein the multimedia content is captured by a camera or microphone of the fitness device).

In the aforementioned embodiments, each set of activity statistics can be assigned a unique identifier. Thus, associating the multimedia content with the statistics can comprise including a unique identifier of statistics in a record associated with the multimedia content.

In step 808, method 800A can comprise posting multimedia content and statistics to a profile associated with a user.

As discussed above, a given user can maintain a profile on the application servers. Such a profile can comprise social media profile and thus include autobiographical data as well as friend connections with other users. Additionally, the application servers can allow a user to “post” on their profile. Such posts can include the recorded multimedia content as well as statistics. In some embodiments, the multimedia content and statistics can be combined into a single image or graphic. For example, the statistics can be overlaid on the recorded multimedia content.

After posting such posts, the application servers can provide an interface (e.g., website, mobile application, fitness device application) that allows users to view another user's posts, including multimedia content and statistics. In some embodiments, method 800A can also allow users to cross-post multimedia content and statistics to third-party social networks (e.g., Facebook®, Twitter®, Instagram®, TikTok®, etc.). By contrast, in other embodiments, method 800A can only allow access to a user's profile within an application associated with the fitness device. Thus, in such embodiments, method 800A provides a “closed” social network limited only to those users using the fitness device.

In one embodiment, the interactions can be filtered by other users. In one embodiment, the multimedia content posted in step 808 can be viewed by other users in, for example, a social network. As part of this operation, other users can filter available multimedia content using a variety of filters. For example, other users can filter or sort multimedia content based on the age of the posting user, the gender of the posting user, the post date, whether the posting user is a friend of the other user, etc. In one embodiment, some filters may be hardcoded. For example, a user may only see posts from their friends and may only be able to apply filters to their friends' multimedia content.

In some embodiments, method 800A can further comprise allowing a user to add music or other audio to the multimedia content. In such an embodiment, method 800A can provide a library of licensed audio to the user and allow the user to select a licensed audio track (e.g., song) to include with the multimedia content and/or statistics. In such an embodiment, method 800A can comprise converting the multimedia content and/or statistics to a video format and mixing the audio into the video format.

In step 810, method 800A can comprise recording interactions with the multimedia content and/or statistics.

In one embodiment, each “friend” of a user can interact with the multimedia content posted in step 808. In one embodiment, method 800A can limit the scope of interactions to only positive interactions (e.g., “hearts,” “likes,” “thumbs up,” etc.) to discourage negative interactions. In one embodiment, an interaction can include comments. In such an embodiment, method 800A can employ sentiment analysis natural language processing libraries to filter negative sentiments and thus ensure only positive comments are posted. In some embodiments, comments may be omitted entirely. In some embodiments, a set of positive emojis can be provided to allow users to respond using such emojis. In one embodiment, these interactions can be associated with the multimedia content and saved for display when displaying the multimedia content.

FIG. 8B is a flow diagram illustrating a method for generating a year in review multimedia object according to some embodiments of the disclosure.

In step 812, method 800B can comprise segmenting multimedia content by month.

In one embodiment, the multimedia content segmented in step 812 comprises the multimedia content recorded and associated as described in the description of FIG. 8A, the details of which are not repeated again herein. In brief, each item of multimedia content is recorded at a given time and is associated with a set of statistics. In one embodiment, method 800B includes a preliminary step (not illustrated) of filtering the multimedia content based on an activity type (e.g., identifying all multimedia content and statistics associated with spin classes or rowing workouts).

After filtering, method 800B segments the multimedia content by month or another similar period (e.g., quarterly, weekly, daily, etc.) and then proceeds to process each segment. In one embodiment, the number of segments is dynamic and can be adjusted. As will be discussed, method 800B selects one item of multimedia content per segment. Thus, method 800B can increase or decrease the segmenting interval to correspondingly increase or decrease the amount of multimedia content selected.

In step 814, method 800B can comprise selecting top multimedia content per month.

In one embodiment, method 800B can analyze each multimedia content for a given month and order the multimedia content for a given month by a preconfigured statistic. For example, method 800B can sort the multimedia content based on a total distance or total calories burned. As discussed, while a month is used as a segment duration, other segment durations (e.g., week, quarter, day) can be used. Further, the specific statistic used to sort the multimedia content is not limited, and other statistics can be used as sort criteria. In one embodiment, after sorting, method 800B can select one or more of the top multimedia content from the sorted list. In one embodiment, method 800B comprises selecting a single item of multimedia content (e.g., the first in the sorted list). In other embodiments, more than one item of multimedia content can be selected.

In step 816, method 800B can comprise generating aggregated multimedia content based on the top multimedia content.

In one embodiment, method 800B can combine the selected top multimedia content into a single video file. In one embodiment, the selected top multimedia content is ordered based on a segment ordering. For example, the multimedia content can be arranged based on month order. In other embodiments, the top multimedia content can be ordered based on the underlying statistics. For example, the top multimedia content can be ordered in an ascending metric (e.g., total output) order. In some embodiments, formats other than video can be used. For example, method 800B can generate a slide show, image gallery, or other multimedia presentation. In some embodiments, the multimedia content can be augmented with the corresponding statistics (as discussed previously) prior to generating the aggregated multimedia content. In some embodiments, step 816 can further comprise adding additional audio (e.g., music) to the aggregated multimedia content.

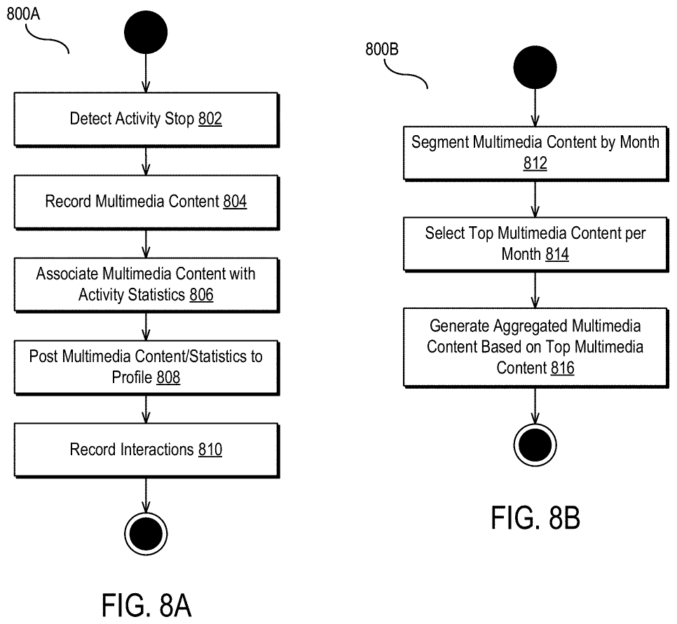

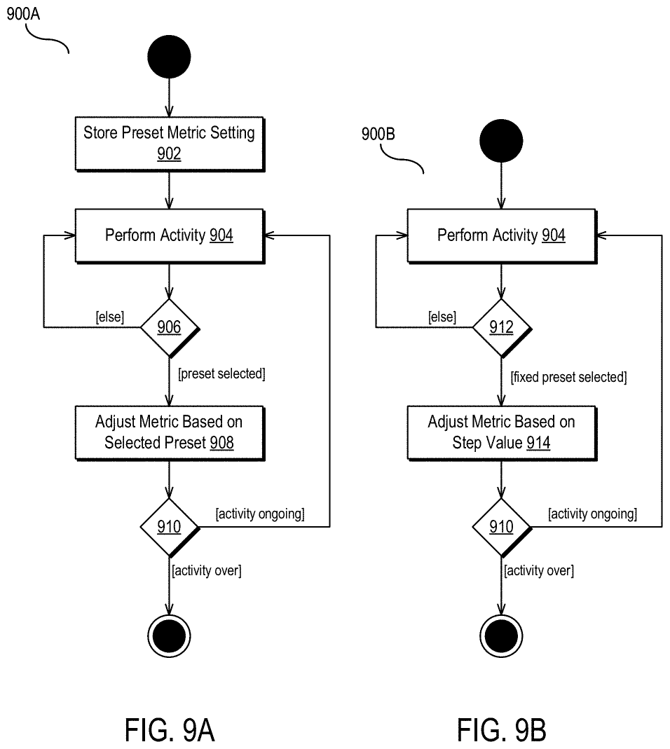

FIG. 9A is a flow diagram illustrating a method for adjusting a metric level according to some embodiments of the disclosure.

In block 902, method 900A comprises storing a preset metric level. In one embodiment, the metric level can comprise a resistance level of an exercise or spin bike, although other metrics may be utilized as discussed previously.

In one embodiment, a preset metric level comprises a numeric value. In one embodiment, the numeric value can comprise an absolute value (e.g., a fixed value) of the metric level. Examples of such numeric values were provided previously in the description of FIGS. 1B, 1C, and 1D.

In one embodiment, the preset metric level can be defined by a manufacturer. In one embodiment, the preset metric level is constant and not changeable. In another embodiment, the preset metric level can be defined by a user. In such an embodiment, a manufacturer can provide an initial value that may be changed by the user. In one embodiment, although only a single preset metric level is described in the illustrated method 900A, any number of preset metric levels can be implemented. In one embodiment, the preset metric levels are stored on a mobile computing device (e.g., mobile phone or tablet device) and are communicatively coupled to exercise equipment via a physical (e.g., wired) or wireless (e.g., Wi-Fi, Bluetooth®, etc.). In some embodiments, the preset metric levels can be stored in a remote computing device and thus persisted on both a tablet device as well as a cloud system. In some embodiments, the cloud system can synchronize the preset metric level across multiple such tablet devices. In some embodiments, the preset metric is further associated with a label (e.g., “Flatroad,” “Hill,” “Climb”). Alternatively, or in conjunction with the foregoing, the preset metric can be associated with an icon.

In block 904, method 900A can comprise performing an activity. As previously discussed, an activity can comprise a fitness class or activity performed on fitness devices such as a stationary or spin bike, a rowing machine, treadmill, etc. In either event, the fitness device can include a digitally controllable component that can adjust the operation of the fitness device. For example, the fitness device can include a magnetic or fluid resistance system that can be adjusted via digital commands. As another example, the fitness device can have a mechanical incline/decline system for inclining and declining the fitness device. In block 904, these systems are configured according to a default metric level at the beginning of the activity. For example, a spin bike can be configured to operate at a preset initial resistance level.

In block 906, method 900A determines if a preset is selected. If not, method 900A continues to perform the activity in block 904 using the current metric level. If so, method 900A adjusts the metric level based on the selected preset metric level in block 908.

In one embodiment, a user of the fitness device can select a preset metric level by selecting an icon on a UI presented on a touch screen device communicatively coupled (either via wired or wireless connection) to the fitness device. Examples of such Uls are provided in FIGS. 1B, 1C, and 1D and the disclosure of those Uls are included herein in their entirety. As one example, a user can select an icon to select one of the multiple preset resistance levels of a spin bike.

In one embodiment, each control is attached to a corresponding event handler. In one embodiment, each control is attached to a separate event handler, while in other embodiments, the controls are all connected to a single event handler and include a type property to distinguish among the controls. When a user selects the control, the event handler is called, and a type of the control can be identified based on a manually passed parameter or by a caller identification. Method 900A can then use the type parameter to determine the corresponding preset metric level. As discussed, this preset metric level can be user-customized. After loading the preset metric level into memory, method 900A can adjust the current metric level of the fitness device to match the preset metric level. In some embodiments, method 900A can simply transmit the preset metric level to the corresponding mechanical system implementing the metric level. In other embodiments, method 900A can read the current metric level, update the metric level to match the preset metric level, and return the updated value to the mechanical system. A pseudocode example of such an event handler is provided below.

```
function handlePresetTouchUp(event) {
  let newResistance = 0;
  switch(event.id) {
    case ‘flatland’:
      newResistance = Settings find(‘presets.flatland’)
      break;
    case ‘hill’:
      newResistance = Settings find(‘presets.hill’)
      break;
    case ‘climb’:
      newResistance = Settings find(‘presets.climb’)
      break;
  }
  if (newResistance > 0) {
    Equipment.magneticResistance = newResistance
  }
}
```

In this pseudocode, the function handlePresetTouchUp is called each time a control is activated for a given preset. The parameter (event) includes an id property that identifies the calling control. In the illustrated code, three controls are used as depicted in FIGS. 1B, 1C, and 1D. Next, the code initializes a temporary variable (newResistance) and then determines which control called the event handler via the switch block. The event handler queries a settings object which comprises a source of settings data for the system and finds a corresponding setting using a theoretical query language. Next, the event handler determines if a valid (e.g., greater than zero) preset metric level is received from the settings and sets a magneticResistance property of an Equipment object to the retrieved resistance value. Here, the Equipment object and magneticResistance property may be used to physically change the magnetic or fluid resistance of the fitness device hardware. In some embodiments, method 900A can further comprise increasing or decreasing a current resistance (Equipment.magneticResistance) over a period of time (e.g., one second) until it matches the new resistance, thus preventing an immediate increase in resistance and providing a gradual change in the same.

In block 910 the method determines if the activity is still ongoing. If so, method 900A continues to execute steps 904, 906, and 908 while the activity is ongoing. If not, method 900A ends.

FIG. 9B is a flow diagram illustrating a method for adjusting a metric level according to some embodiments of the disclosure.

In block 904, method 900B can comprise performing an activity. Details of this step are provided in connection with FIG. 9A and are not repeated herein.

In block 912, method 900B can comprise determining if a fixed preset is selected. In one embodiment, the fixed preset can correspond to a fixed metric level such as a fixed resistance level of an exercise or spin bike, although other metrics may be utilized as discussed. In one embodiment, a fixed metric level comprises a numeric value. In one embodiment, the numeric value comprises a relative increase or decrease in a current metric level. For example, the preset metric level can comprise a +5 or −5 increment or decrement, respectively, of a metric level. Examples of such numeric values were provided previously in the description of FIGS. 1B, 1C, and 1D.

In one embodiment, the fixed metric level can be defined by a manufacturer. In one embodiment, the fixed metric level is constant and not changeable. In another embodiment, the fixed metric level can be defined by a user. In such an embodiment, a manufacturer can provide an initial value that may be changed by the user. In one embodiment, although only a single fixed metric level is described in the illustrated method 900B, any number of fixed metric levels can be implemented. In one embodiment, the fixed metric levels are stored on a mobile computing device (e.g., mobile phone or tablet device) and are communicatively coupled to exercise equipment via a physical (e.g., wired) or wireless (e.g., Wi-Fi, Bluetooth®, etc.). In some embodiments, the fixed metric levels can be stored in a remote computing device and thus persisted on both a tablet device as well as a cloud system. In some embodiments, the cloud system can synchronize the fixed metric levels across multiple such tablet devices.

In one embodiment, a user of the fitness device can select a fixed preset by selecting an icon on a UI presented on a touch screen device communicatively coupled (either via wired or wireless connection) to the fitness device. Examples of such UIs are provided in FIGS. 1B, 1C, and 1D and the disclosure of those Uls are included herein in their entirety. As one example, a user can select an icon to select one of multiple preset resistance increases of a spin bike.

In one embodiment, each fixed preset control is attached to a corresponding event handler. In one embodiment, each fixed preset control is attached to a separate event handler while in other embodiments, the fixed preset controls are all connected to a single event handler and include a type property to distinguish among the fixed preset controls. When a user selects the control, the event handler is called and a type of the control can be identified based on a manually passed parameter or by a caller identification. Method 900B can then use the type parameter to determine the corresponding fixed metric level. As discussed, this fixed metric level can be user-customized. After loading the fixed metric level into memory, method 900B can adjust the current metric level of the fitness device to match the increase corresponding to the fixed metric level. In some embodiments, method 900B can read the current metric level, update the current metric level to match the current metric level offset by the fixed metric level, and return the updated value to the mechanical system. A pseudo-code example of such an event handler is provided below.

```
function handleFixedMetricIncrease(event) {
  let currentResistance = Equipment.magneticResistance;
  switch(event.caller) {
    case ‘increase’:
      Equipment.magneticResistance +=
        Settings find( ‘presets.fixed_metric_change.increase’)
      break;
    case ‘decrease’:
      Equipment.magneticResistance +=
        Settings.find(‘presets.fixed_metric_change.decrease’)
      break;
  }
}
```

In this pseudocode, the function handleFixedMetricIncrease is called each time a fixed preset control is activated for a given preset. The parameter (event) includes a caller property that identifies the calling fixed preset control. In the illustrated code, two controls (increase or decrease) are used as depicted in FIGS. 1B, 1C, and 1D. Next, the code determines which control (increase or decrease) called the event handler via the switch block. In each case statement, the event handler queries a Settings object which comprises a source of setting data for the system and finds a corresponding fixed preset setting (e.g., +5 or −5) using a theoretical query language. Next, the event increases or decreases magneticResistance property of an Equipment object based on the retrieved resistance value. In some embodiments, method 900B can further comprise increasing or decreasing a current resistance (Equipment.magneticResistance) over a period of time (e.g., one second) until it affects the increase or decrease in resistance, thus preventing an immediate increase in resistance and providing a gradual change in the same.

In block 910, the method determines if the activity is still ongoing. If so, method 900B continues to execute steps 904, 912, and 914 while the activity is ongoing. If not, method 900B ends.

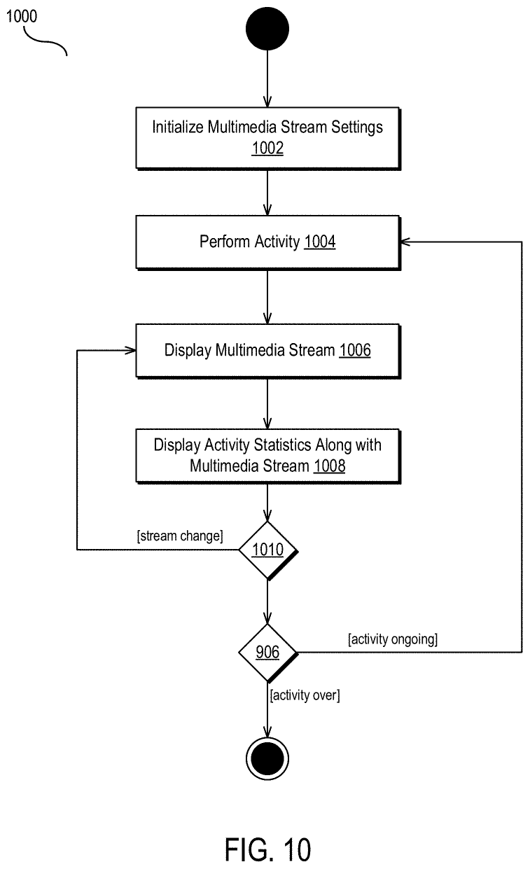

FIG. 10 is a flow diagram illustrating a method for displaying streaming media content during an activity according to some embodiments of the disclosure.

In step 1002, method 1000 can comprise initializing multimedia stream settings.

In one embodiment, a multimedia stream refers to a stream of multimedia content (e.g., video and/or audio and/or images) received over a network from a remote computing device such as a media server. In some embodiments, multimedia streams can be provided by third-party multimedia service providers such as Netflix®, Hulu®, Spotify® or similar types of multimedia providers.

In one embodiment, method 1000 can store login credentials and other settings for such multimedia service providers. In some embodiments, login credentials can comprise username/password combinations, authentication tokens, or other forms of identification mechanisms.

In one embodiment, method 1000 can initiate a multimedia stream by authenticating to a third-party multimedia service provider and displaying a UI allowing a user to select a multimedia stream. For example, a UI for a music multimedia service provider may provide access to a list of songs available for streaming as well as a library of the user's saved songs, etc. Similarly, a video multimedia service provider may provide a user interface allowing a user to select episodes of television programs or individual movies. In response to the selection of an icon or image representing a stream (e.g., a song element or a movie poster), method 1000 can initiate a stream with the third-party multimedia service provider. For example, method 1000 can initiate a WebRTC or similar stream session with a designated media server.

As will be seen, in some embodiments, method 1000 can delay the stream start until after an activity is started. Thus, step 1002 may comprise the initialization and user interaction portions of initiating a multimedia stream.

In step 1004, method 1000 can comprise performing an activity. As previously discussed, an activity can comprise a fitness class or activity performed on fitness devices such as a stationary or spin bike, a rowing machine, treadmill, etc. During such an operation, the fitness device records and caches statistics as previously described. Examples of statistics or metrics are depicted in FIGS. 1A, 1B, 1C, and 1D and include, but are not limited to, metrics such as cadence, resistance, distance, speed, calories, output, etc.

In step 1006, method 1000 can comprise displaying the multimedia stream.

In one embodiment, method 1000 is executed in a “freestyle” mode wherein no pre-recorded or live activity content is transmitted to the fitness device implementing the method. For example, in some embodiments, the user may select such a mode which does not place any limit on the type and amount of activity that can be performed. Thus, in these embodiments, UI 100 may be modified to remove the leaderboard 104, leaving only the status panel 106, heads-up display 108, and main window 102. In such an embodiment, main window 102 can be used to allow a user to select a multimedia stream and can also be used to stream the multimedia stream. In other embodiments, a new section of the UI can be used to display the multimedia stream. For example, a floating window can be used to overlay the multimedia stream on top of the UI. In some embodiments, the multimedia stream may have various controls to control the playback as well as load a new multimedia stream.

In step 1008, method 1000 can comprise simultaneously displaying activity statistics along with the multimedia stream. As discussed, in some embodiments, the multimedia stream can be display in main window 102. In such an embodiment, the statistics can remain displayed in status panel 106. In some embodiments, the multimedia stream can be full screen and each of sections 106 and 108 can be overlaid on top of the multimedia stream. In any event, while the user is performing the activity the user can simultaneously view his or her metrics as well as the multimedia stream. Further, the user can manipulate the UI to select new multimedia streams (e.g., new songs).

In some embodiments, method 1000 may further provide for voice controls to allow a user to switch multimedia streams without the need for physical interaction with the UI. In some embodiments, method 1000 may further provide for automatically changing multimedia streams. For example, method 1000 can automatically select new songs based on the cadence or other metrics of the user. In some embodiments, method 1000 can further comprise automatically adjusting the playback of the multimedia streams in response to the metrics. For example, method 1000 can detect that a user has stopped exercising due to a significant decrease in power output, speed, etc. In response, method 1000 can temporarily pause a multimedia stream and display a prompt requesting the user confirm they are still using the fitness device. Alternatively, or in conjunction with the foregoing, method 1000 can automatically resume a multimedia stream upon detecting that the metrics have returned to a normal setting (e.g., an average setting of the activity or other user's global performance) or have started again in general. In this manner, method 1000 can pause and resume multimedia streams based on the presence of a user without the need for the user to manually start and stop multimedia streams.

In step 1010, method 1000 determines if a change in the multimedia stream has occurred. In one embodiment, a change can occur when a user proactively changes the multimedia stream (e.g., selects a new song) via a UI. Alternatively, or in conjunction with the foregoing, a change can occur automatically when a multimedia stream is placed with a new multimedia stream (e.g., playing the next song or next episode of an album or television program, respectively). When a change occurs, method 1000 executes steps 1006 and 1008 for the new multimedia stream.

If no change occurs, method 1000 continuously executes steps 1004, 1006, 1008, and 1010 while the activity is ongoing. In step 1012, method 1000 determines whether the activity has ended. As discussed in previous figures, method 1000 can determine that an activity has ended when a condition is met (e.g., a distance or time has been reached) or when the user affirmatively ends the activity (e.g., by canceling or ending the workout).

The present disclosure has been described with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in some embodiments” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure has been described with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure, a non-transitory computer readable medium (or computer-readable storage medium/media) stores computer data, which data can include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

In the preceding specification, various example embodiments have been described with reference to the accompanying drawings. However, it will be evident that various modifications and changes may be made thereto, and additional embodiments may be implemented without departing from the broader scope of the disclosed embodiments as set forth in the claims that follow. The specification and drawings are accordingly to be regarded in an illustrative rather than restrictive sense.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to the desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. The present disclosure can refer to the action and processes of a computer system, or similar electronic computing device, that manipulates and transforms data represented as physical (electronic) quantities within the computer system's registers and memories into other data similarly represented as physical quantities within the computer system memories or registers or other such information storage systems.

The present disclosure also relates to an apparatus for performing the operations herein. This apparatus can be specially constructed for the intended purposes, or it can include a general-purpose computer selectively activated or reconfigured by a computer program stored in the computer. Such a computer program can be stored in a computer-readable storage medium, such as but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

The algorithms and displays presented herein are not inherently related to any particular computer or other apparatus. Various general-purpose systems can be used with programs in accordance with the teachings herein, or it can prove convenient to construct a more specialized apparatus to perform the method. The structure for a variety of these systems will appear as set forth in the description below. In addition, the present disclosure is not described with reference to any particular programming language. It will be appreciated that a variety of programming languages can be used to implement the teachings of the disclosure as described herein.

The present disclosure can be provided as a computer program product or software that can include a machine-readable medium having stored thereon instructions, which can be used to program a computer system (or other electronic devices) to perform a process according to the present disclosure. A machine-readable medium includes any mechanism for storing information in a form readable by a machine (e.g., a computer). In some embodiments, a machine-readable (e.g., computer-readable) medium includes a machine (e.g., a computer) readable storage medium such as read-only memory (“ROM”), random access memory (“RAM”), magnetic disk storage media, optical storage media, flash memory components, etc.

In this description, various functions and operations are described as being performed by or caused by computer instructions to simplify the description. However, those skilled in the art will recognize what is meant by such expressions is that the functions result from the execution of the computer instructions by one or more controllers or processors, such as a microprocessor. Alternatively, or in combination, the functions and operations can be implemented using special-purpose circuitry, with or without software instructions, such as using Application-Specific Integrated Circuit (ASIC) or Field-Programmable Gate Array (FPGA). Embodiments can be implemented using hardwired circuitry without software instructions or in combination with software instructions. Thus, the techniques are limited neither to any specific combination of hardware circuitry and software nor to any particular source for the instructions executed by the data processing system.

In the foregoing specification, embodiments of the disclosure have been described with reference to specific example embodiments thereof. It will be evident that various modifications can be made thereto without departing from the broader spirit and scope of embodiments of the disclosure as set forth in the following claims. The specification and drawings are, accordingly, to be regarded in an illustrative sense rather than a restrictive sense.

What is claimed is:

1. A method comprising:
detecting, by a processor communicatively coupled to a fitness device, a stop of an activity, the activity comprising a physical activity performed on the fitness device;
recording multimedia content of a user of the fitness device in response to the stop of the activity;
associating the multimedia content with at least one recorded metric, the at least one recorded metric recorded during the performance of the activity; and
transmitting the multimedia content and the at least one recorded metric to a remote computing device.

2. The method of claim 1, wherein the activity comprises an activity selected from the group consisting of a spin class, cycling class, rowing class, and workout class.

3. The method of claim 1, wherein the multimedia content comprises content selected from the group consisting of video, image, or audio content.

4. The method of claim 1, wherein associating the multimedia content with at least one recorded metric comprises generating statistics describing the user's performance during the activity; assigning a unique identifier to the statistics; and associating the unique identifier with the multimedia content.

5. The method of claim 1, wherein recording multimedia content comprises recording the multimedia content using a camera communicatively coupled to the processor.

6. The method of claim 1, wherein recording multimedia content comprises recording the multimedia content using a mobile device of the user.

7. The method of claim 1, further comprising generating aggregated multimedia content based on historical multimedia content, the historical multimedia content including the multimedia content.

8. A method comprising:
storing, by a computing device communicatively coupled to a fitness device, a plurality of metric settings, each of the plurality of metric settings comprising a numeric value and associated with a physical metric of the fitness device controllable by the computing device;
displaying, by the computing device, a plurality of controls, each control associated with a corresponding metric setting and situated near a status panel displayed on a screen of the computing device;
detecting, by the computing device, a selection of a selected control, the selected control comprising one of the plurality of controls; and
adjusting, by the computing device, the physical metric of the fitness device in response to detecting the selection.

9. The method of claim 8, wherein the plurality of metric settings includes a preset metric setting, the preset metric setting comprising a numeric value to set the physical metric to.

10. The method of claim 8, wherein the plurality of metric settings includes a fixed preset setting, the fixed preset setting comprising a numerical value to increment or decrement the physical metric by.

11. The method of claim 8, wherein the physical metric comprises a resistance of the fitness device.

12. The method of claim 8, wherein displaying a plurality of controls comprises displaying a plurality of text labels in the plurality of controls.

13. The method of claim 12, wherein displaying a plurality of controls comprises displaying a plurality of icons in the plurality of controls.

14. The method of claim 8, wherein displaying a plurality of controls comprises displaying a plurality of icons in the plurality of controls.

15. The method of claim 8, further comprising modifying the plurality of metric settings in response to a user input.

16. The method of claim 8, where adjusting the physical metric of the fitness device comprises gradually increasing or decreasing the physical metric.

17. The method of claim 8, further comprising synchronizing the plurality of metric settings with a remote computing device.

18. A method comprising:
initializing a multimedia stream;
recording metrics associated with an activity performed on a fitness device; and
simultaneously displaying the multimedia stream and the metrics while a user performs the activity.

19. The method of claim 18, wherein the multimedia stream comprises a stream selected from the group consisting of a video, audio, or image stream.

20. The method of claim 18, further comprising stopping a multimedia stream in response to detecting a change in the metrics.
